(19) United States
(12) Patent Application Publication
(10) Pub. No.: US 2024/0347139 A1
(43) Pub. Date: Oct. 17, 2024

(54) METHOD, APPARATUS, AND DEVICE FOR PREDICTING ADVERSE DRUG-DRUG INTERACTIONS, AND READABLE STORAGE MEDIUM

(71) Applicant: University of Electronic Science and Technology of China, Chengdu (CN)

(72) Inventors: Jiajing ZHU, Chengdu (CN); Yongguo LIU, Chengdu (CN); Zhi CHEN, Chengdu (CN); Yun ZHANG, Chengdu (CN); Qiaoqin LI, Chengdu (CN); Xin LU, Chengdu (CN)

(73) Assignee: University of Electronic Science and Technology of China, Chengdu (CN)

(21) Appl. No.: 18/533,616

(22) Filed: Dec. 8, 2023

(30) Foreign Application Priority Data
Apr. 11, 2023 (CN) 202310379566.9

(51) Int. Cl. G16C 20/10; G16C 20/50

(52) U.S. Cl. CPC G16C 20/10; G16C 20/50

(57) ABSTRACT

Disclosed are a method, an apparatus, and a device for predicting adverse drug-drug interactions, and a readable storage medium, and relates to the technical field of pharmaceutical research and development. An objective of the present application is to provide the method, apparatus, and device for predicting adverse drug-drug interactions, and the readable storage medium for solving the above problems. To achieve the above objective, a technical solution adopted by the present application is as follows: acquiring first information; constructing an adverse drug-drug interaction prediction model according to the first information; acquiring second information, where the second information is molecular structure information of two drugs to be predicted; and outputting predicted adverse drug-drug interactions with the second information as an input into the adverse drug-drug interaction prediction model.

### CROSS-REFERENCE TO RELATED APPLICATIONS

This application claims priority to Chinese Patent Application No. 202310379566.9, filed on Apr. 11, 2023, the content of which is incorporated herein by reference in its entirety.

### TECHNICAL FIELD

The present application relates to the technical field of pharmaceutical research and development, in particular to a method, an apparatus, and a device for predicting adverse drug-drug interactions, and a readable storage medium.

### BACKGROUND

The existing research on prediction of adverse drug-drug interactions is mainly based on a knowledge-based method and a similarity-based method. Existing adverse drug-drug interaction prediction models mostly establish an adverse drug-drug interaction relation based on features of molecular structures of drugs. The above methods usually use PubChem substructure fingerprints of drug molecules for converting SMILES molecular formulas of the molecular structures of the drugs into their binary code. Based on this, the adverse drug-drug interactions can be predicted by computing the binary code similarities between two drugs. However, the applicant found that the above methods still have the following defects: first, binary code information of the molecular structures of the drugs is incomplete, which leads to insufficient code representation of the planar features of the molecular structures of the drugs; and second, during prediction of the adverse interactions, numerous non-critical factors that have little influence on the adverse drug-drug interactions are considered, complicating the binary code of the planar features of the molecular structures of the drugs, failing to predict key factors causing adverse interactions and influencing prediction accuracy.

### SUMMARY

An objective of the present application is to provide a method, an apparatus, and a device for predicting adverse drug-drug interactions, and a readable storage medium for solving the above problems. In order to achieve the above objective, a technical solution is adopted by the present application as follows.

In a first aspect, the present application provides a method for predicting adverse drug-drug interactions. The method includes the following steps:
acquiring first information, where the first information includes molecular structure information of two drugs and the adverse drug-drug interaction data, and the molecular structure information of the drugs includes molecular structures of the drugs and molecular substructures of the drugs;
constructing an adverse drug-drug interaction prediction model according to the first information as follows: constructing the code of planar features of the molecular structures according to the molecular substructures of two drugs, performing feature selection for the code of the planar features of the molecular structures of two drugs, and constructing the adverse drug-drug interaction prediction model based on the code of selected planar features of the molecular structures of two drugs and the adverse drug-drug interaction data, where the feature selection is to select critical molecular substructures of the drug that cause the adverse interactions;
acquiring second information, where the second information is molecular structure information of two drugs to be predicted; and
outputting predicted adverse drug-drug interactions with the second information as an input into the adverse drug-drug interaction prediction model.

In a second aspect, the present application further provides an apparatus for predicting adverse drug-drug interactions. The apparatus includes:
a first acquiring module configured to acquire first information, where the first information includes molecular structure information of two drugs and the adverse drug-drug interaction data, and the molecular structure information of the drugs includes molecular structures of the drugs and molecular substructures of the drugs;
a first constructing module configured to construct an adverse drug-drug interaction prediction model according to the first information as follows: constructing the code of planar features of the molecular structures according to the molecular substructures of two drugs, performing feature selection for the code of the planar features of the molecular structures of two drugs, and constructing the adverse drug-drug interaction prediction model based on the code of selected planar features of the molecular structures of two drugs and the adverse drug-drug interaction data, where the feature selection is to select critical molecular substructures of the drug that cause the adverse interactions;
a second acquiring module configured to acquire second information, where the second information is molecular structure information of two drugs to be predicted; and
a solving module configured to output predicted adverse drug-drug interactions with the second information as an input into the adverse drug-drug interaction prediction model.

In a third aspect, the present application further provides a device for predicting adverse drug-drug interactions. The device includes:
a memory configured to store a computer program; and
a processor configured to implement steps of the method for predicting adverse drug-drug interactions when executing the computer program.

In a fourth aspect, the present application further provides a readable storage medium. The readable storage medium stores a computer program, where the computer program implements steps of the method for predicting adverse drug-drug interactions when executed by a processor.

The present application has the beneficial effects: according to the present application, the adverse drug-drug interaction prediction model is constructed, such that representation of the code of planar features of the molecular structures of the drugs is improved. The critical molecular substructures of the drug influencing the adverse drug-drug interactions are revealed, and the potential adverse drug-drug interactions are explored, such that data support is provided for experimental study of the adverse drug-drug interactions, occurrences of adverse drug-drug interactions events are reduced, and significance for enhancing safety of drug use is achieved.

Other features and advantages of the present application will be set forth in the following description, and will partially become apparent in the description, or can be learned by implementing examples of the present application. The objective and other advantages of the present application can be achieved and obtained through structures particularly indicated in the description, the claims and accompanying drawings.

### BRIEF DESCRIPTION OF THE DRAWINGS

To describe technical solutions in examples of the present application more clearly, accompanying drawings required in the examples of the present application will be briefly described below. It should be understood that the following accompanying drawings show merely some examples of the present application, and should not be considered as limitation to the scope accordingly. A person of ordinary skill in the art can still derive other relevant accompanying drawings from these accompanying drawings without creative efforts.

FIG. 1 is a schematic flowchart of a method for predicting adverse drug-drug interactions according to an example of the present application.

FIG. 2 is a schematic structural diagram of an apparatus for predicting adverse drug-drug interactions according to an example of the present application.

FIG. 3 is a schematic structural diagram of a device for predicting adverse drug-drug interactions according to an example of the present application.

FIG. 4 is a schematic diagram showing modeling of relative position of molecular substructures in aspirin according to an example of the present application.

FIG. 5 is a schematic diagram showing modeling of relative position of molecular substructures in warfarin according to an example of the present application.

FIG. 6 is a schematic diagram of a code of planar features of molecular structure of aspirin according to an example of the present application.

FIG. 7 is a schematic diagram of the code of planar feature of molecular structure of warfarin according to an example of the present application.

FIG. 8 shows a framework for an adverse drug-drug interaction prediction model based on feature selection according to an example of the present application.

FIG. 9 is a schematic flowchart of step S200 according to an example of the present application.

FIG. 10 is a schematic flowchart of step S201 according to an example of the present application.

FIG. 11 is a schematic flowchart of step S202 according to an example of the present application.

FIG. 12 is a schematic flowchart of step S203 according to an example of the present application.

FIG. 13 is a schematic flowchart of step S204 according to an example of the present application.

### DETAILED DESCRIPTION OF THE EMBODIMENTS

To make the objectives, technical solutions, and advantages of the examples of the present application clearer, the technical solutions in the examples of the present application will be clearly and completely described below with reference to accompanying drawings in the examples of the present application. Apparently, the described examples are some examples rather than all examples of the present application. In general, assemblies, described and shown in the accompanying drawings herein, of the examples of the present application can be arranged and designed in various configurations. As a result, the detailed description, in the accompanying drawings below, of the examples of the present application is not intended to limit the protection scope claimed by the present application, but merely denotes selected examples of the present application. Based on the examples of the present application, all the other examples derived by those of ordinary skill in the art without creative efforts shall fall within the protection scope of the present application.

It should be noted that similar reference numerals and letters indicate similar items in the following accompanying drawings, and once defined in one accompanying drawing, an item is unnecessary to be further defined and explained in subsequent accompanying drawings. In addition, in the description of the present application, the terms “first”, “second”, etc., are merely used for distinguishing the description and cannot be understood as indicating or implying relative importance.

An adverse drug interaction is considered as “a harmful or unrelated interaction that occurs when a drug is taken for prevention, diagnosis, disease treatment or physiological function regulation”. The adverse interactions mainly include drug side effects, toxic effects, allergic interactions and specific interactions. Critical factors for the adverse drug interactions include drug use errors, drug management technology implementation errors, apparatus failures, and improper monitoring of patients after drug use. The combined use of drugs of various kinds will also increase the risk of the adverse drug interactions. For example, adverse interactions such as skin discomfort, nervous system discomfort, muscle toxicity and liver toxicity will be caused when statins are combined with antihypertensive drugs, antidiabetic drugs, antiviral drugs, and anticoagulant drugs.

A molecular structure of a drug describes the arrangement manner of drug molecules on planes, and a molecular substructure of the drug denotes the atomic group with a certain function in the molecular structure of the drug.

### Example 1

This example provides a method for predicting adverse drug-drug interactions.

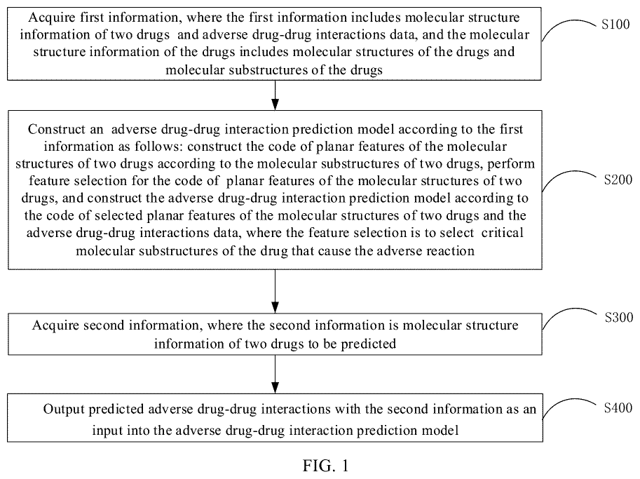

With reference to FIG. 1, the method includes S100, S200, S300 and S400 as shown in the figure.

The method for predicting adverse drug-drug interactions includes:

S100: first information is acquired, where the first information includes molecular structure information of two drugs and adverse drug-drug interaction data, and the molecular structure information of the drugs includes molecular structures of the drugs and molecular substructures of the drugs.

In S100, adverse drug-drug interaction data are collected specifically from TWOSIDES database. The TWOSIDES database record adverse interactions caused by the combination of two drugs. The molecular structure information of the drugs is collected from DrugBank database. SMILES molecular formula of the molecular structure of the drugs described in DrugBank database is transformed into a molecular structural diagram of the drugs with package redk toolkit in R language.

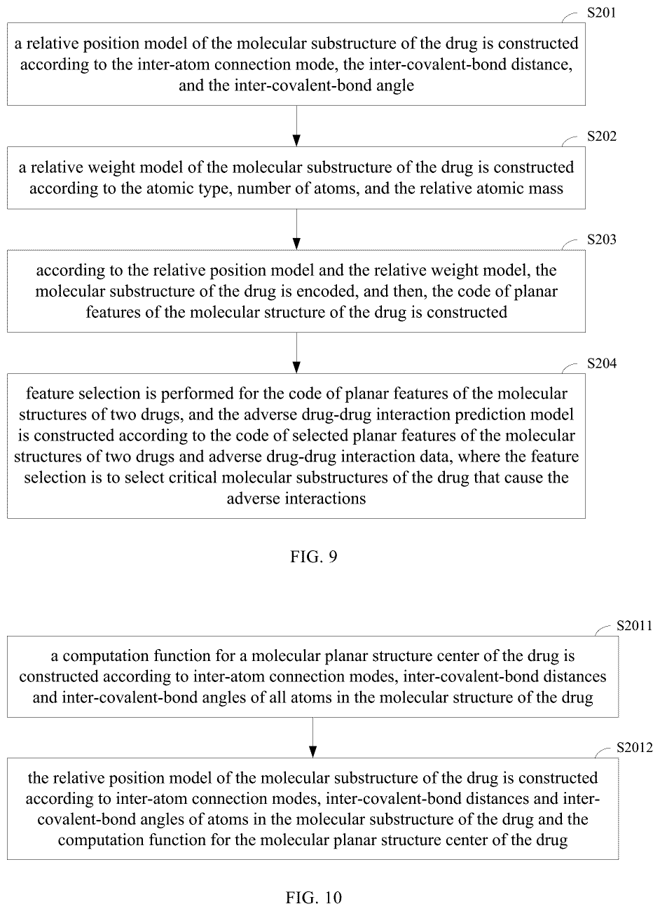

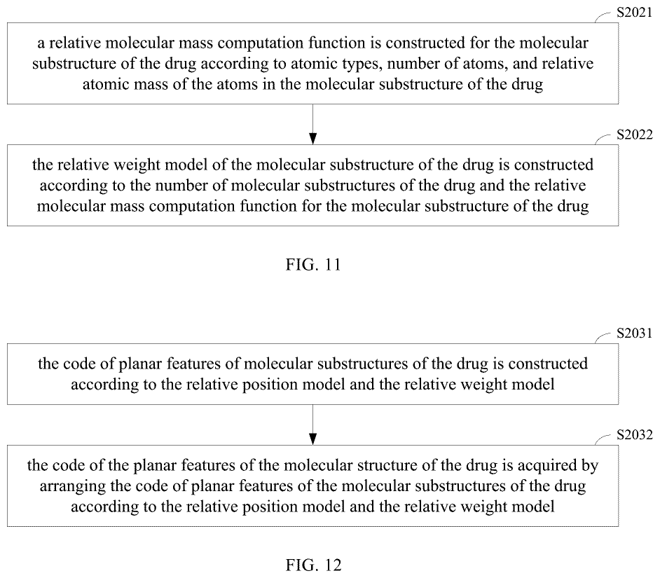

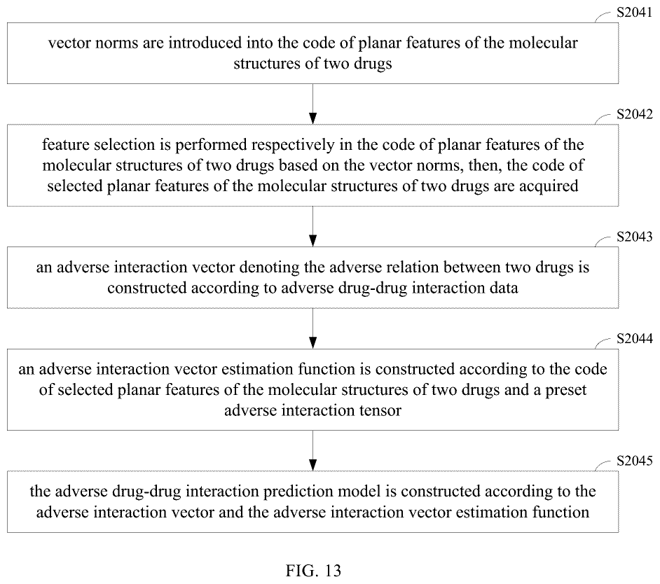

As shown in FIG. 9-13:
S200: an adverse drug-drug interaction prediction model is constructed according to the first information as follows: the code of planar features of the molecular structures of two drugs are constructed according to the molecular substructures of two drugs, feature selection is performed for the code of planar features of the molecular structures of two drugs, and the adverse drug-drug interaction prediction model is constructed according to code of selected planar features of the molecular structures of two drugs and the adverse drug-drug interaction data, where the feature selection is to select critical molecular substructures of the drug that cause the adverse interaction.

The molecular structure information of the drug includes inter-atom connection mode, inter-covalent-bond distance, inter-covalent-bond angle, atomic type, number of atoms, and relative atomic mass, and S200 that the code of planar features of the molecular structures of the drugs are constructed according to the molecular substructures of the drugs specifically includes:
S201: a relative position model of the molecular substructure of the drug is constructed according to the inter-atom connection mode, the inter-covalent-bond distance, and the inter-covalent-bond angle.
S201 specifically includes:
S2011: a computation function for a molecular planar structure center of the drug is constructed according to inter-atom connection modes, inter-covalent-bond distances and inter-covalent-bond angles of all atoms in the molecular structure of the drug.
S2012: the relative position model of the molecular substructure of the drug is constructed according to inter-atom connection modes, inter-covalent-bond distances and inter-covalent-bond angles of atoms in the molecular substructure of the drug and the computation function for the molecular planar structure center of the drug.
S201 will be further described below with aspirin and warfarin as examples.

The inter-atom connection modes include chemical bonds, such as a single bond, a double bond, a triple bond, and a benzene ring. Since the connection modes, connection distances and inter-covalent-bond angles between atoms are critical factors influencing the relative position of the molecular substructure of the drug, the applicant considered that the structural center of an overall molecular structure of the drug can be computed at first, and the relative position of the molecular substructure of the drug can be then determined according to a positional relation between the structural center and the molecular substructure of the drug.

A planar rectangular coordinate system is constructed according to the inter-atom connection modes, inter-covalent-bond distances, and inter-covalent-bond angles of all atoms in the molecular structure of the drug.

According to a trigonometric function, the inter-covalent-bond angle of all atoms in the molecular structure information of the drug are mapped to a horizontal coordinate axis and a vertical coordinate axis of the planar rectangular coordinate system, to acquire a relative position of each atom on the planar rectangular coordinate system.

According to the relative position of each atom in the molecular structure of the drug on the planar rectangular coordinate system, the computation function for the molecular planar structure center of the drug is constructed based on the trigonometric function.

The relative position model of the molecular substructure of the drug is constructed according to the inter-atom connection modes, inter-covalent-bond distances, and inter-covalent-bond angles of the atoms in the molecular substructure of the drug, the computation function for the molecular planar structure center of the drug and a relative position computation function. The relative position computation function includes a horizontal and vertical offset computation function and an offset angle computation function of each atom in the molecular substructure of the drug relative to the molecular planar structure center of the drug.

With the planar structures of aspirin and warfarin as examples, relative positions of molecular substructures in aspirin and warfarin are computed based on inter-atom connection modes, connection distances, and inter-covalent-bond angles.

As shown in FIG. 4, a schematic diagram showing modeling of the relative position of molecular substructures in aspirin is shown. The molecular substructures in aspirin are expressed as αᵢ, i=1, . . . , N, and N denotes the number of the molecular substructures in aspirin.

In aspirin, the set of inter-atom connection modes is expressed as L^{α}, the set of connection distances is expressed as D^{α}, and the set of inter-covalent-bond angles is expressed as A^{α}. Then, the planar structure center (X^{α}, Y^{α}) of aspirin may be expressed as follows:

$$( {X}^{α} , {Y}^{α} ) = f ( {L}^{α} , {D}^{α} , {A}^{α} ) \qquad ( 1 )$$

where ƒ denotes a computation function for the molecular planar structure center of the drug.

According to the inter-atom connection modes, inter-covalent-bond distances, and inter-covalent-bond angles in aspirin, a planar rectangular coordinate system may be constructed. The molecular structure center is determined by computing a complex trigonometric function between atoms. In the molecular substructure αᵢ of the drug, the set of inter-atom connection modes is expressed as L^{α}^{ᵢ}, the set of connection distances is expressed as D^{α}^{ᵢ}, and the set of inter-covalent-bond angles is expressed as A^{α}^{ᵢ}. By combining the planar structural center (X^{α}, Y^{α}) of aspirin, the relative position of the molecular substructure αᵢ of the drug may be expressed as follows:

$$( {X}^{{α}_{i}} , {Y}^{{α}_{i}} ) = g ( {L}^{{α}_{i}} , {D}^{{α}_{i}} , {A}^{{α}_{i}} , ( {X}^{α} , {Y}^{α} ) ) \qquad ( 2 )$$

where g denotes a computation function for relative position of the molecular substructure of the drug. Relative position coordinates (X^{α}^{ᵢ}, Y^{α}^{ᵢ}) of each molecular substructure of the drug are determined similarly according to the structure center position (X^{α}, Y^{α}) and the inter-atom connection modes, connection distances, and inter-covalent-bond angles in the molecular substructure of the drug.

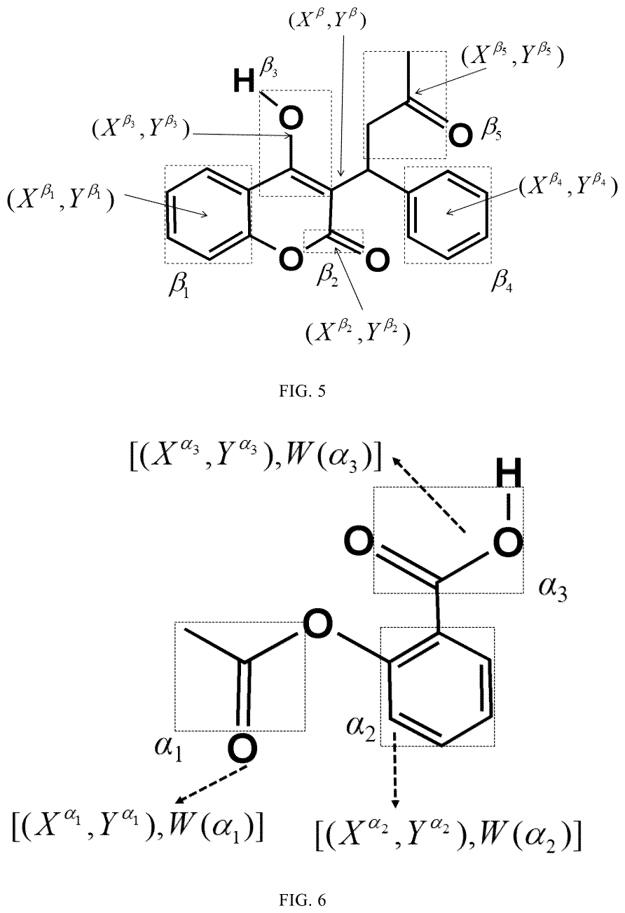

As shown in FIG. 5, a schematic diagram showing modeling of the relative position of molecular substructures in warfarin is shown. The molecular substructures in warfarin are expressed as βⱼ, j=1, . . . , M, M denotes the number of the molecular substructures in warfarin. In warfarin, the set of inter-atom connection modes is expressed as L^{β}, the set of connection distances is expressed as D^{β}, and the set of inter-covalent-bond angles is expressed as A^{β}. Then, the planar structure center (X^{β}, Y^{β}) of warfarin may be expressed as follows:

$$( {X}^{β} , {Y}^{β} ) = f ( {L}^{β} , {D}^{β} , {A}^{β} ) \qquad ( 3 )$$

where ƒ denotes a computation function for a molecular planar structure center of the drug. Based on that, by combining the planar structure center (X^{β}, Y^{β}) of warfarin, the inter-atom connection mode L^{β}^{ⱼ}, the connection distance D^{β}^{ⱼ}, and the inter-covalent-bond angle A^{β}^{ⱼ} of the molecular substructure βⱼ of the drug, the relative position of the molecular substructure βⱼ of the drug may be expressed as follows:

$$( {X}^{{β}_{j}} , {Y}^{{β}_{j}} ) = g ( {L}^{{β}_{j}} , {D}^{{β}_{j}} , {A}^{{β}_{j}} , ( {X}^{β} , {Y}^{β} ) ) \qquad ( 4 )$$

where g denotes a computation function for relative position of the molecular substructure of the drug.
S202: a relative weight model of the molecular substructure of the drug is constructed according to the atomic type, number of atoms, and the relative atomic mass.
S202 specifically includes:
S2021: a relative molecular mass computation function is constructed for the molecular substructure of the drug according to atomic types, number of atoms, and relative atomic mass of the atoms in the molecular substructure of the drug.
S2022: the relative weight model of the molecular substructure of the drug is constructed according to the number of molecular substructures of the drug and the relative molecular mass computation function for the molecular substructure of the drug.

In the molecular substructure of the drug, the weight of the molecular substructure of the drug is influenced by the atomic type, number of atoms, and relative atomic mass of elements. The periodic table of elements records the atomic number, atomic type, family number and relative atomic mass of each element. The relative atomic mass R refers to 1/12 of the atomic mass of a carbon-12 (C-12) as the standard. The ratio of true mass of any atom to 1/12 of the mass of the carbon-12 atom is referred to as relative atomic mass of the atom.

In the step, with planar structures of aspirin and warfarin as examples, relative weights of molecular substructures in aspirin and warfarin are computed based on the atomic type, number of atoms, and relative atomic mass of the element.

The relative molecular mass of molecular substructure αᵢ in aspirin and βⱼ in warfarin can be expressed as H(αᵢ)=ΣₖCₖ^{α}^{ᵢ}Rₖ, and H(βⱼ)=Σₖ Cₖ^{β}^{ⱼ}Rₖ, respectively, where Cₖ^{α}^{ᵢ} denotes the number of the k-th atom in molecular substructure αᵢ of the drug, and Rₖ denotes relative atomic mass of the k-th atom.

Further, in order to eliminate the influence of some elements that have large relative atomic mass and low occurrence frequencies on computation of the relative weight of the molecular substructure of the drug and improve weight contribution of the number of atoms to the computation of the relative molecular mass of the molecular substructure of the drug, a functional relation between the number of the atoms in the molecular substructure of the drug and the relative molecular mass of the molecular substructure of the drug is constructed when the relative molecular mass of the molecular substructure of the drug is computed. Given the molecular substructure αᵢ of the drug, C^{α}^{ᵢ}=Σₖ Cₖ^{α}^{ᵢ} denotes the number of atoms in the molecular substructure αᵢ of the drug, and a function y(C^{α}^{ᵢ}) denotes a contribution value of the number of atoms C^{α}^{ᵢ} to computation of the relative molecular mass of the molecular substructure of the drug. Then, relative molecular mass H(αᵢ) and H(βⱼ) of molecular substructure αᵢ and βⱼ of the drug may be expressed as follows:

$$H ( {α}_{i} ) = y ( {C}^{{α}_{i}} ) {\sum }_{k} {C}_{k}^{{α}_{i}} {R}_{k} \qquad ( 5 )$$

$$H ( {β}_{j} ) = y ( {C}^{{β}_{j}} ) {\sum }_{k} {C}_{k}^{{β}_{j}} {R}_{k} \qquad ( 6 )$$

The relative molecular weights of the molecular substructure αᵢ and βⱼ of the drug may be expressed as follows:

$$W ( {α}_{i} ) = \frac{H ( {α}_{i} )}{{\sum }_{l = 1}^{N} H ( {α}_{l} )} \qquad ( 7 )$$

$$W ( {β}_{j} ) = \frac{H ( {β}_{j} )}{{\sum }_{l = 1}^{M} H ( {β}_{l} )} \qquad ( 8 )$$

During modeling of the relative position of the molecular substructure of the drug, the positional center of the molecular substructure of the drug is determined according to the inter-atom connection mode and connection distance, and besides, the center of gravity of the molecular substructure of the drug is computed by combining the relative weight of the molecular substructure of the drug. An integration strategy is designed to implement relative position code with relative weight information, such that spatial dimensions of the code of the molecular substructure of the drug are reduced, and spatial complexity of the code of planar features of the molecular structure of drug is reduced.
S203: according to the relative position model and the relative weight model, the molecular substructure of the drug is encoded, and then, the code of planar features of the molecular structure of the drug is constructed.
S203 specifically includes:
S2031: the code of planar features of molecular substructures of the drug is constructed according to the relative position model and the relative weight model.
S2032: the code of the planar features of the molecular structure of the drug is acquired by arranging the code of planar features of the molecular substructures of the drug according to the relative position model and the relative weight model.

With the planar structures of aspirin and warfarin as examples described above, molecular substructure information of the drug is specifically encoded based on the relative position and the relative weight of the molecular substructure of the drug, thereby improving representation of the code of planar feature of the molecular structure of the drug. The relative position and the relative weight of the molecular substructure of the drug are used to describe features of the molecular substructure of the drug, thereby encoding the planar feature of the molecular substructure of the drug. Given the relative position (X^{α}^{ᵢ}, Y^{α}^{ᵢ}) and relative weight W(αᵢ) of the molecular substructure αᵢ of the drug, the code of the planar features of molecular substructure αᵢ of the drug can be expressed as follows:

$$[ ( {X}^{{α}_{i}} , {Y}^{{α}_{i}} ) , W ( {α}_{i} ) ] \qquad ( 9 )$$

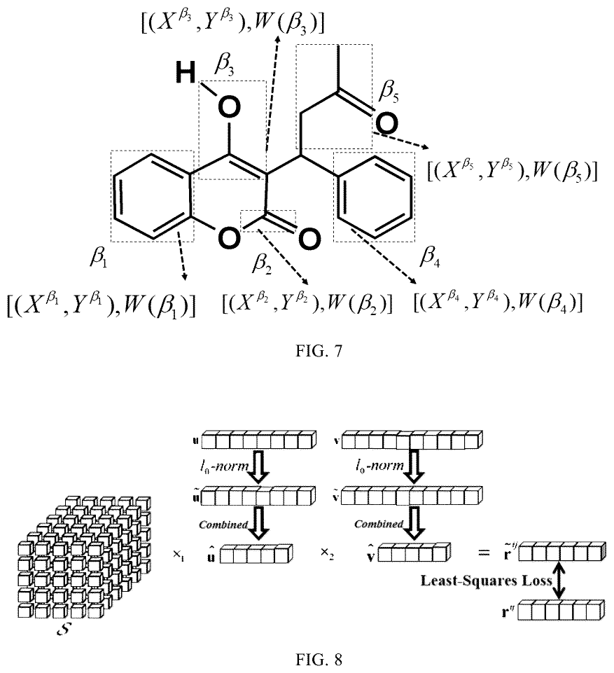

As shown in FIG. 6, a schematic diagram of the code of planar features of molecular structure of aspirin is shown. FIG. 7 is a schematic diagram of the code of planar features of molecular structure of warfarin. FIG. 6 shows only the case where i is 3, and FIG. 7 shows only the case where j is 4. The code of planar features of molecular substructure αᵢ in aspirin can be expressed as [(X^{α}^{ᵢ}, Y^{α}^{ᵢ}), W(αᵢ)], where (X^{α}^{ᵢ}, Y^{α}^{ᵢ}) denotes the relative position of αᵢ, and W(αᵢ) denotes the relative weight of at. The code of planar features of molecular substructure βⱼ in warfarin can be expressed as [(X^{β}^{ⱼ}, Y^{β}^{ⱼ}), W(βⱼ)], where (X^{β}^{ⱼ}, Y^{β}^{ⱼ};) denotes the relative position of βⱼ, and W(βⱼ) denotes the relative weight of βⱼ. During encoding of the feature of the molecular structure of the drug, relative positions and relative weights of molecular substructures of the drug are arranged in order.

Based on that, the code of planar features of the molecular structure of aspirin can be expressed as follows:

$$u = [ \{ ( {X}^{{α}_{1}} , {Y}^{{α}_{1}} ) , W ( {α}_{1} ) \} , \{ ( {X}^{{α}_{2}} , {Y}^{{α}_{2}} ) , W ( {α}_{2} ) \} , … , \{ ( {X}^{{α}_{N}} , {Y}^{{α}_{N}} ) , W ( {α}_{N} ) \} ] \qquad ( 10 )$$

The code of planar features of the molecular structure of warfarin can be expressed as follows:

$$v = [ \{ ( {X}^{{β}_{1}} , {Y}^{{β}_{1}} ) , W ( {β}_{1} ) \} , \{ ( {X}^{{β}_{2}} , {Y}^{{β}_{2}} ) , W ( {β}_{2} ) \} , … , \{ ( {X}^{{β}_{M}} , {Y}^{{β}_{M}} ) , W ( {β}_{M} ) \} ] \qquad ( 11 )$$

S204: feature selection is performed for the code of planar features of the molecular structures of two drugs, and the adverse drug-drug interaction prediction model is constructed according to the code of selected planar features of the molecular structures of two drugs and adverse drug-drug interaction data, where the feature selection is to select critical molecular substructures of the drug that cause the adverse interactions.
S204 specifically includes:
S2041: vector norms are introduced into the code of planar features of the molecular structures of two drugs.
S2042: feature selection is performed respectively in the code of planar features of the molecular structures of two drugs based on the vector norms, then, the code of selected planar features of the molecular structures of two drugs are acquired.
S2043: an adverse interaction vector denoting the adverse relation between two drugs is constructed according to adverse drug-drug interaction data.
S2044: an adverse interaction vector estimation function is constructed according to the code of selected planar features of the molecular structures of two drugs and a preset adverse interaction tensor.
S2045: the adverse drug-drug interaction prediction model is constructed according to the adverse interaction vector and the adverse interaction vector estimation function.

The adverse drug-drug interaction prediction model is constructed based on feature selection, such that dimensions of the code of features of the molecular structure of the drug are reduced, and critical molecular substructures of the drug influencing the adverse drug-drug interactions are revealed. With aspirin and warfarin as examples in the step, a feature selection model is constructed based on the code of planar features of the molecular structures of two drugs, and adverse interactions are predicted.

Since the number of molecular substructures in the drugs varies from each other, it is necessary to construct a consistence strategy to eliminate the difference in the number of molecular substructures of different drugs, so as to achieve consistent modeling of adverse drug-drug interactions. On the other hand, since the adverse drug-drug interactions are merely influenced by a small number of critical molecular substructures of the drug generally, the remaining molecular substructures of the drug, as accompanying information of the critical molecular substructures of the drug, have little influence on adverse drug-drug interactions. Therefore, based on the code of the planar features of molecular structure of the drug, the adverse drug-drug interaction prediction model based on feature selection is proposed in this step. By introducing a vector 10 norm, the number of the molecular substructures of the drug that influences the adverse drug-drug interactions is constrained, and the critical molecular substructures of the drug that influence the adverse drug-drug interactions are explored. By introducing the vector norm, non-selected molecular substructures of the drug are removed, and the code of selected planar features of the molecular structure of the drug is acquired.

Specifically, based on the code u and v of planar features of the molecular structures of aspirin and warfarin, the vector l₀ norm is introduced, the critical molecular substructures of the drug causing the adverse interaction are selected from the code u and v respectively, and code vectors ú and \acute{v} with selected features are acquired. The vector l₀ norm is used to select non-zero elements from the vector and 0 is assigned to non-selected elements. In FIG. 8, the number of non-zero elements in the vector is 5, that is, C critical molecule substructures (C=5) are selected from the code u and v, planar features of the molecular structures of aspirin and warfarin respectively, the corresponding position of the remaining non-selected molecular substructure of the drug is set to 0, and the code ũ and \tilde{v} of selected planar features of the molecular structures of the drugs are acquired. On this basis, the non-selected molecular substructures of the drug are removed, and only the selected molecular substructures of the drug are kept, that is, the non-zero elements in ũ and \tilde{v} are kept, and vectors ũ and \tilde{v} are acquired.

1188258 groups of adverse drug-drug interaction data are collected in total, and 59377 adverse interaction drug pairs are involved, covering N*=567 kinds of drugs and K*=258 kinds of adverse interactions. Basic common drugs and adverse interactions are covered. Given a drug set D={d₁, d₂, . . . , d_{N*}}, for the adverse interactions between the drugs dᵢ and dⱼ, a vector r^{ij}∈{0,1}^{K*} is constructed for denoting the adverse interactions relation between dᵢ and dⱼ. If the K-th adverse interaction is caused between dᵢ and dⱼ, r_{K}^{ij}=1; otherwise, r_{K}^{ij}=0, where K∈{1,258}.

In order to construct an adverse interaction model between aspirin and warfarin, and explore the potential relation of the code û and \hat{v} of selected planar features of the molecular structure of the drug with the adverse interaction vector r^{ij}, adverse interaction tensor [symbol] ∈ [symbol] is introduced, and an estimated value \tilde{r}^{ij} of the vector r^{ij} is as follows:

$${\tilde{r}}^{ij} = 𝒮 \times {}_{1} \hat{u} \times {}_{2} \hat{v} \qquad ( 12 )$$

where ×₁ denotes the product of first order of the adverse interaction tensor and the vector, and ×₂ denotes the product of second order of the adverse interaction tensor and the vector.

Based on that, the objective function of the adverse drug-drug interaction prediction model may be written as follows:

$${\sum }_{{‖ {r}^{ij} ‖}_{0} \neq 0} {‖ {r}^{ij} - {\tilde{r}}^{ij} ‖}_{2}^{2} \qquad ( 13 )$$

where ∥r^{ij}∥₀ denotes vector l₀ norm, and ∥r^{ij}∥≠0 denotes that r^{ij} is a non-zero vector.

Based on vector l₀ norm optimization and a low-rank canonical polyadic (CP) decomposition method of a high-order tensor, vector l₀ norm is transformed into an optimal convex approximate l₁ norm, and tensor [symbol] is decomposed into the sum of tensors of rank 1, thereby, estimation of the adverse drug-drug interactions r^{ij} is realized. Finally, an implicit parameter of the model is iteratively optimized by using a stochastic gradient descent method, so as to update the parameters of the model. As shown in FIG. 8, a framework for adverse drug-drug interaction prediction model based on feature selection is shown. In FIG. 8, the norm is used to specify the number of critical molecular substructures of the drug selected from the code vector of planar feature of the molecular structure of the drug, Combined denotes a non-zero element in a combined vector, and Least-squares Loss denotes a least squares loss function.

S300: second information is acquired, where the second information is molecular structure information of two drugs to be predicted.

Given molecular structure information of any two drugs, the adverse drug-drug interactions can be predicted. SMILES molecular formulas of molecular structures of the drugs to be predicted for adverse drug-drug interactions are extracted from DrugBank database at first, and then, the SMILES molecular formulas of the molecular structures of the drugs are transformed into molecular structural diagrams of the drugs by package redk toolkit in R language.

S400: the second information is used as the input to the adverse drug-drug interaction prediction model, and the predicted adverse drug-drug interactions are output.

Using the adverse drug-drug interaction prediction model proposed in this present application, the relative position and the relative weight of the molecular substructure of the drugs are modeled, the molecular substructure information of the drugs is encoded based on the relative position and the relative weight of the molecular substructure of the drugs. Then, the planar features of the molecular structure of the drug is encoded. Based on the optimized tensor [symbol] and the code of the planar features of the molecular structure of the drug, the adverse drug-drug interaction vector is computed with formula (12), and the adverse drug-drug interactions are predicted.

By using the provided method for predicting adverse drug-drug interactions based on the code of planar features of the molecular structure of the drug, the adverse drug-drug interactions can be predicted, and some potential adverse drug-drug interactions can be revealed, such that data support is provided for study of the adverse drug-drug interactions and safety of new drugs based on biological experimental methods. In an actual application, the present application is adopted to predict the adverse drug-drug interactions as follows:

An adverse drug-drug interaction prediction experiment 1: the present application is used to predict the adverse interactions between venlafaxine and olanzapine, and molecular structure information of venlafaxine and olanzapine is used as an input. Through the present application, it is predicted that the combination of venlafaxine and the olanzapine will cause adverse interactions such as loss of consciousness, paranoia and sleepiness when used together.

An adverse drug-drug interaction prediction experiment 2: the present application is used to predict the adverse interactions between valsartan and amlodipine, and molecular structure information of valsartan and amlodipine is used as an input. Through the present application, it is predicted that the combination of valsartan and amlodipine will cause adverse interactions such as dizziness, diarrhea, nausea, and palpitations when used together.

According to the present application, the relative position model and the relative weight model of the molecular substructures of two drugs are combined to encode the molecular structures of the drugs, such that representation of the code of planar features of the molecular structure of the drug is improved.

The adverse drug-drug interaction prediction model is constructed, and feature selection is performed for the code of planar features of the molecular structures of two drugs. The feature selection is to select the critical molecular substructures of the drug that cause the adverse interaction, such that the critical molecular substructures of the drug influencing the adverse drug-drug interactions are revealed, and numerous non-critical factors that have little influence on the adverse drug-drug interactions are removed. The potential adverse drug-drug interactions are explored, data support is provided for experimental study of adverse drug-drug interactions, occurrences of adverse drug-drug interaction events are reduced, and significance for promotion of safety of drug use is achieved.

### Example 2

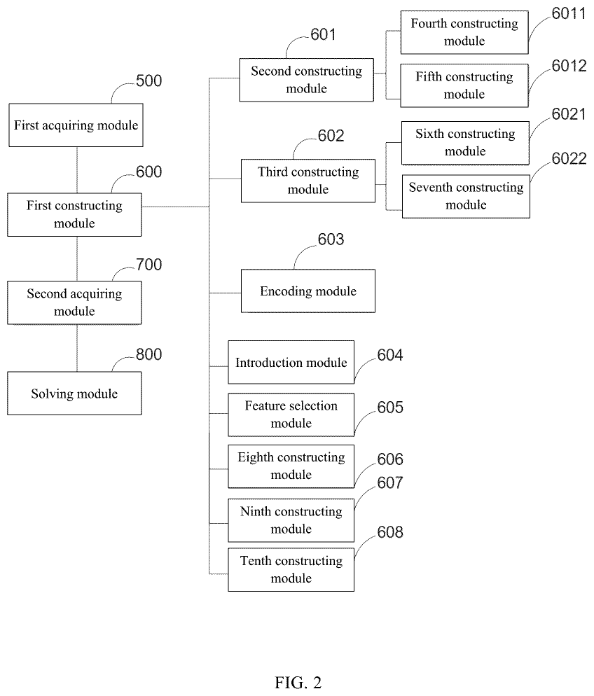

As shown in FIG. 2, an apparatus for predicting adverse drug-drug interactions is provided according to this example. The apparatus includes:
a first acquiring module 500 configured to acquire first information, where the first information includes molecular structure information of two drugs and adverse drug-drug interaction data, and the molecular structure information of the drugs includes molecular structures of the drugs and molecular substructures of the drugs;
a first constructing module 600 configured to construct an adverse drug-drug interaction prediction model according to the first information as follows: constructing the code of planar features of the molecular structures of two drugs according to the molecular substructures of two drugs, performing feature selection for the code of planar features of the molecular structures of two drugs, and constructing the adverse drug-drug interaction prediction model according to the code of selected planar features of the molecular structures of two drugs and the adverse drug-drug interaction data, where the feature selection is to select the critical molecular substructures of the drug that cause the adverse interactions;
a second acquiring module 700 configured to acquire second information, where the second information is molecular structure information of two drugs to be predicted; and
a solving module 800 configured to output the predicted adverse drug-drug interactions with the second information as an input into the adverse drug-drug interaction prediction model.

The first constructing module 600 includes:
a second constructing module 601 configured to construct a relative position model of the molecular substructure of the drug according to inter-atom connection mode, inter-covalent-bond distance and inter-covalent-bond angle;
a third constructing module 602 configured to construct a relative weight model of the molecular substructure of the drug according to atomic type, number of atoms, and relative atomic mass; and
an encoding module 603 configured to encode the molecular substructure of the drug and construct the code of planar features of the molecular structure of the drug according to the relative position model and the relative weight model.

The second constructing module 601 includes:
a fourth constructing module 6011 configured to construct a computation function for the molecular planar structure center of the drug according to inter-atom connection modes, inter-covalent-bond distances, and inter-covalent-bond angles of all atoms in the molecular structure of the drug; and
a fifth constructing module 6012 configured to construct the relative position model of the molecular substructure of the drug according to inter-atom connection modes, inter-covalent-bond distances and inter-covalent-bond angles of atoms in the molecular substructure of the drug and the computation function for the molecular planar structure center of the drug.

The third constructing module 602 includes:
a sixth constructing module 6021 configured to construct a relative molecular mass computation function for the molecular substructure of the drug according to atomic types, number of atoms, and relative atomic mass of the atoms in the molecular substructure of the drug; and
a seventh constructing module 6022 configured to construct the relative weight model of the molecular substructure of the drug according to the number of molecular substructures of the drug and the relative molecular mass computation function for the molecular substructure of the drug.

The first constructing module 600 further includes:
an introduction module 604 configured to introduce vector norms into the code of planar features of the molecular structures of two drugs;
a feature selection module 605 configured to select features separately in the code of planar features of the molecular structures of two drugs based on the vector norms and acquire the code of selected planar features of the molecular structures of two drugs;
an eighth constructing module 606 configured to construct an adverse interaction vector denoting adverse relation between two drugs according to the adverse drug-drug interaction data;
a ninth constructing module 607 configured to construct an adverse interaction vector estimation function according to the code of planar features of the molecular structures of two drugs and a preset adverse interaction tensor; and
a tenth constructing module 608 configured to construct the adverse drug-drug interaction prediction model according to the adverse interaction vector and the adverse interaction vector estimation function.

It should be noted that with respect to the apparatus in the above example, specific ways in which the modules execute operations have been described in detail in the examples relating to the method, and will not be described in detail herein.

### Example 3

Corresponding to the above method example, this example further provides a device for predicting adverse drug-drug interactions. The device for predicting adverse drug-drug interactions described below and the method for predicting adverse drug-drug interactions described above can refer to each other correspondingly.

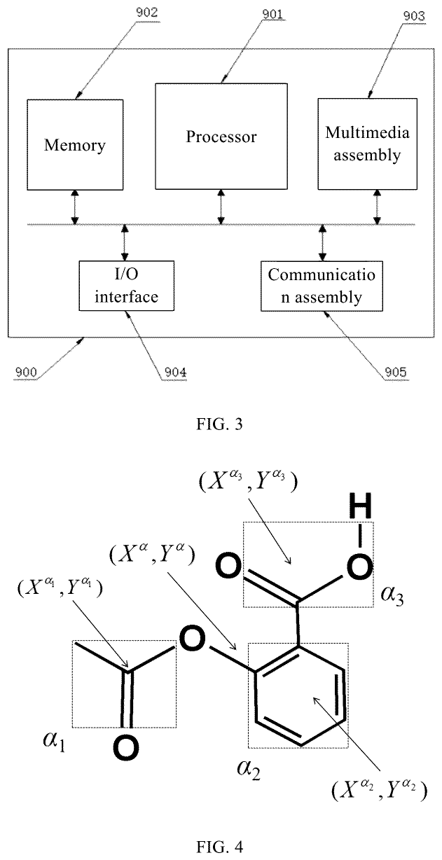

FIG. 3 is a block diagram of a device 900 for predicting adverse drug-drug interactions according to an illustrative example. As shown in FIG. 3, the device 900 for predicting adverse drug-drug interactions may include a processor 901 and a memory 902. The device 900 for predicting adverse drug-drug interactions may further include one or more of a multimedia assembly 903, an I/O interface 904 and a communication assembly 905.

The processor 901 is configured to control an overall operation of the device 900 for predicting adverse drug-drug interactions, so as to implement all or some steps in the method for predicting adverse drug-drug interactions. The memory 902 is configured to store various types of data to support the operation by the device 900 for predicting adverse drug-drug interactions. These data may include, for example, operating instructions for any application or method for the device 900 for predicting adverse drug-drug interactions, as well as application-related data, such as contact data, messages, pictures, audios and videos. The memory 902 may be implemented by means of any type of volatile or non-volatile storage device, or their combinations, for example, a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic disk or an optical disk. The multimedia assembly 903 may include a screen and an audio assembly. The screen may be, for example, a touch screen, and the audio assembly is configured to output and/or input an audio signal. For example, the audio assembly may include a microphone, and the microphone is configured to receive an external audio signal. The received audio signal may be further stored in the memory 902 or transmitted via the communication assembly 905. The audio assembly may further include at least a speaker for outputting audio signals. The I/O interface 904 provides an interface between the processor 901 and other interface module that may be a keyboard, a mouse, a button, etc. These buttons may be virtual buttons or physical buttons. The communication assembly 905 is configured for wired or wireless communication between the device 900 for predicting adverse drug-drug interactions and other devices. For wireless communication, such as Wi-Fi, Bluetooth, near field communication (NFC), 2G, 3G or 4G, or a combination of one or more of such items, the corresponding communication assembly 905 may include: a Wi-Fi module, a Bluetooth module, and an NFC module.

In an illustrative example, the device 900 for predicting adverse drug-drug interactions may be implemented by one or more application specific integrated circuits (ASIC), digital signal processors (DSP), digital signal processing devices (DSPD), programmable logic devices (PLD), field programmable gate arrays (FPGA), controllers, microcontrollers, microprocessors or other electronic components, and is configured to execute the method for predicting adverse drug-drug interactions.

In another illustrative example, a computer-readable storage medium is further provided. The computer-readable storage medium includes program instructions, where the program instructions are executed by the processor to implement steps of the method for predicting adverse drug-drug interactions. For example, the computer-readable storage medium may be the memory 902 including the program instructions. The program instructions may be executed by the processor 901 of the device 900 for predicting adverse drug-drug interactions to implement the method for predicting adverse drug-drug interactions.

### Example 4

Corresponding to the above method example, this example further provides a readable storage medium. The readable storage medium described below and the method for predicting adverse drug-drug interactions described above can refer to each other correspondingly.

The readable storage medium is provided. The readable storage medium stores a computer program, where the computer program is executed by a processor to implement steps of the method for predicting adverse drug-drug interactions of the method example.

The readable storage medium may specifically include a USB flash drive, a mobile hard disk drive, a read-only memory (ROM), a random access memory (RAM), a diskette or optical disk, etc., which may store program codes.

The above examples are merely preferred examples of the present application and are not intended to limit the present application, and for those skilled in the art, the present application can be modified and changed in various forms. Any modification, equivalent substitution, improvement, etc. made within the spirit and principles of the present application shall fall within the protection scope of the present application.

What are described above are merely specific embodiments of the present application, but the protection scope of the present application is not limited such embodiments. Any change or substitution that can be easily conceived by any person of ordinary skill in the art within the technical scope disclosed by the present application should fall within the protection scope of the present application. The protection scope of the present application should be subject to a protection scope of the claims accordingly.

What is claimed is:

1. A method for predicting adverse drug-drug interactions, comprising:
acquiring first information, wherein the first information comprises molecular structure information of two drugs and adverse drug-drug interaction data, and the molecular structure information of the drugs comprises molecular structures of the drugs and molecular substructures of the drugs;
constructing an adverse drug-drug interaction prediction model according to the first information, comprising:
constructing a code of planar features of the molecular structures of two drugs according to the molecular substructures of two drugs;
performing feature selection for the code of planar features of the molecular structures of two drugs, the feature selection being used to select critical molecular substructures of the drug that cause the adverse interaction; and
constructing the adverse drug-drug interaction prediction model according to the code of selected planar features of the molecular structures of two drugs and the adverse drug-drug interaction data;
acquiring second information, wherein the second information is molecular structure information of two drugs to be predicted; and
outputting predicted adverse drug-drug interactions with the second information as an input into the adverse drug-drug interaction prediction model.

2. The method for predicting adverse drug-drug interactions according to claim 1, wherein the molecular structure information of the drug comprises an inter-atom connection mode, an inter-covalent-bond distance, an inter-covalent-bond angle, an atomic type, a number of atoms, and relative atomic mass, and constructing a code of planar features of the molecular structures of the drugs according to the molecular substructures of the drugs comprises:
constructing a relative position model of the molecular substructure of the drug according to the inter-atom connection mode, the inter-covalent-bond distance, and the inter-covalent-bond angle;
constructing a relative weight model of the molecular substructure of the drug according to the atomic type, the number of atoms, and the relative atomic mass; and
encoding the molecular substructure of the drug and constructing the code of the planar feature of the molecular structure of the drug according to the relative position model and the relative weight model.

3. The method for predicting adverse drug-drug interactions according to claim 2, wherein constructing a relative position model of the molecular substructure of the drug according to the inter-atom connection mode, inter-covalent-bond distance, and the inter-covalent-bond angle and constructing a relative weight model of the molecular substructure of the drug according to the atomic type, the number of atoms, and the relative atomic mass comprise:
constructing a computation function for a molecular planar structure center of the drug according to inter-atom connection modes, inter-covalent-bond distances and inter-covalent-bond angles of all atoms in the molecular structure of the drug;
constructing the relative position model of the molecular substructure of the drug according to inter-atom connection modes, inter-covalent-bond distances and inter-covalent-bond angles of atoms in the molecular substructure of the drug and the computation function for the molecular planar structure center of the drug;
constructing a relative molecular mass computation function for the molecular substructure of the drug according to the atomic type, the number of atoms, and the relative atomic mass of the atoms in the molecular substructure of the drug; and
constructing the relative weight model of the molecular substructure of the drug according to a number of molecular substructures of the drug and the relative molecular mass computation function for the molecular substructure of the drug.

4. The method for predicting adverse drug-drug interactions according to claim 1,
wherein constructing adverse drug-drug interaction prediction model according to the first information comprises:
introducing vector norms into the code of the planar features of the molecular structures of two drugs;
performing feature selection separately in the code of the planar features of the molecular structures of two drugs based on the vector norms and acquiring the code of selected planar features of the molecular structures of two drugs;
constructing an adverse interaction vector denoting the adverse relation between two drugs according to the adverse drug-drug interaction data;
constructing an adverse interaction vector estimation function according to the code of planar features of the molecular structures of two drugs and a preset adverse interaction tensor; and
constructing the adverse drug-drug interaction prediction model according to the adverse interaction vector and the adverse interaction vector estimation function.

5. An apparatus for predicting adverse drug-drug interactions, comprising:
a first acquiring module configured to acquire first information, wherein the first information comprises molecular structure information of two drugs and adverse drug-drug interaction data, and the molecular structure information of the drugs comprises molecular structures of the drugs and molecular substructures of the drugs;
a first constructing module configured to construct an adverse drug-drug interaction prediction model according to the first information, comprising:
construct a code of planar features of the molecular structures of two drugs according to the molecular substructures of two drugs;
perform feature selection for the code of planar features of the molecular structures of two drugs, the feature selection being used to select critical molecular substructures of the drug that cause the adverse interaction; and
construct the adverse drug-drug interaction prediction model according to the code of selected planar features of the molecular structures of two drugs and the adverse drug-drug interaction data;
a second acquiring module configured to acquire second information, wherein the second information is molecular structure information of two drugs to be predicted; and
a solving module configured to output predicted adverse drug-drug interactions with the second information as an input into the adverse drug-drug interaction prediction model.

6. The apparatus for predicting adverse drug-drug interactions according to claim 5, wherein the first constructing module comprises:
a second constructing module configured to construct a relative position model of the molecular substructure of the drug according to the inter-atom connection mode, inter-covalent-bond distance and inter-covalent-bond angle;
a third constructing module configured to construct a relative weight model of the molecular substructure of the drug according to the atomic type, number of atoms, and relative atomic mass; and
an encoding module configured to encode the molecular substructure of the drug and construct the code of planar features of the molecular structure of the drug according to the relative position model and the relative weight model.

7. The apparatus for predicting adverse drug-drug interactions according to claim 6, wherein the first constructing module comprises:
a fourth constructing module configured to construct a computation function for the molecular planar structure center of the drug according to inter-atom connection modes, inter-covalent-bond distances and inter-covalent-bond angles of all atoms in the molecular structure of the drug;
a fifth constructing module configured to construct the relative position model of the molecular substructure of the drug according to inter-atom connection modes, inter-covalent-bond distances and inter-covalent-bond angles of atoms in the molecular substructure of the drug and the computation function for the molecular planar structure center of the drug;
a sixth constructing module configured to construct a relative molecular mass computation function for the molecular substructure of the drug according to atomic types, a number of atoms, and relative atomic mass of the atoms in the molecular substructure of the drug; and
a seventh constructing module configured to construct the relative weight model of the molecular substructure of the drug according to a number of molecular substructures of the drug and the relative molecular mass computation function for the molecular substructure of the drug.

8. The apparatus for predicting adverse drug-drug interactions according to claim 5, wherein the first constructing module comprises:
an introduction module configured to introduce vector norms into the code of planar features of the molecular structures of two drugs;
a feature selection module configured to select features separately in the code of planar features of the molecular structures of two drugs based on the vector norms and acquire the code of selected planar features of the molecular structures of two drugs;
an eighth constructing module configured to construct an adverse interaction vector denoting an adverse relation between two drugs according to the adverse drug-drug interaction data;
a ninth constructing module configured to construct an adverse interaction vector estimation function according to the code of selected planar features of the molecular structures of two drugs and a preset adverse interaction tensor; and
a tenth constructing module configured to construct the adverse drug-drug interaction prediction model according to the adverse interaction vector and the adverse interaction vector estimation function.

9. A device for predicting adverse drug-drug interactions, comprising:
a memory configured to store a computer program; and
a processor configured to implement steps of the method for predicting adverse drug-drug interactions according to claim 1 when executing the computer program.

10. A non-transitory readable storage medium, storing a computer program, wherein the steps of the method for predicting adverse drug-drug interactions according to claim 1 are implemented when the computer program is executed by a processor.
